(19) United States
(12) Patent Application Publication
(10) Pub. No.: US 2023/0001174 A1
(43) Pub. Date: Jan. 5, 2023

(54) Protective Cover for a Medical Device

(71) Applicant: Becton, Dickinson and Company, Franklin Lakes, NJ (US)

(72) Inventors: Shaun Lauer, Portland, OR (US); Benjamin Parker Hopwood, Salt Lake City, UT (US); Curtis H. Blanchard, Riverton, UT (US); Weston F. Harding, Lehi, UT (US)

(21) Appl. No.: 17/852,574

(22) Filed: Jun. 29, 2022

Related U.S. Application Data
(60) Provisional application No. 63/218,167, filed on Jul. 2, 2021.

(51) Int. Cl. A61M 39/10; A61M 25/00

(52) U.S. Cl. CPC A61M 39/1011; A61M 25/0097; A61M 2205/586

(57) ABSTRACT

A medical device may include a distal connector, which may include a first lever arm and a second lever arm. A distal end of the first lever arm and a first end of the second lever arm may include a first protrusion and a second protrusion, respectively. The distal connector may include a blunt cannula disposed between the first lever arm and the second lever arm. The medical device may include a cover coupled to the distal connector and having an annular wall surrounding the blunt cannula. The first protrusion and the second protrusion may contact at least one catch disposed on an outer surface of the annular wall. A proximal end of the first lever arm and a proximal end of the second lever arm may be configured to be pinched together to remove the first protrusion and the second protrusion from the at least one catch.

### CROSS-REFERENCE TO RELATED APPLICATION

The present application claims priority to U.S. Provisional Application Ser. No. 63/218,167, entitled “Protective Cover for a Medical Device”, filed Jul. 2, 2021, the entire disclosure of which is hereby incorporated by reference in its entirety.

### BACKGROUND OF THE INVENTION

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Infusion and blood withdrawal using the catheter may be difficult for several reasons, particularly when an indwelling time of the catheter increases. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample. Sometimes a medical device may be coupled to the catheter assembly and an instrument, such as a guidewire or tube, may be inserted through the catheter assembly to reduce or remove occlusions such as the fibrin sheath or thrombus and extend a life of the catheter. Just prior to use of the medical device and while the medical device is packaged, a distal connector of the medical device may become damaged or contaminated or may pose a danger to a user.

The subject matter claimed herein is not limited to embodiments that solve any disadvantages or that operate only in environments such as those described above. Rather, this background is only provided to illustrate one example technology area where some implementations described herein may be practiced. The protective cover described in the present disclosure may be used with any suitable medical device.

### SUMMARY OF THE INVENTION

The present disclosure relates generally to a cover for a medical device. In some embodiments, a medical device may include a distal connector. In some embodiments, the distal connector may include a first lever arm, which may include a distal end and a proximal end. In some embodiments, the distal end of the first lever arm may include a first protrusion. In some embodiments, the distal connector may include a second lever arm opposing the first lever arm. In some embodiments, the second lever arm may include a distal end and a proximal end. In some embodiments, the distal end of the second lever arm may include a second protrusion. In some embodiments, the distal connector may include a blunt cannula disposed between the first lever arm and the second lever arm.

In some embodiments, the medical device may include a cover coupled to the distal connector. In some embodiments, the cover may include an annular wall surrounding the blunt cannula. In some embodiments, an outer surface of the annular wall may include at least one catch. In some embodiments, the first protrusion and the second protrusion may contact the at least one catch. In some embodiments, the proximal end of the first lever arm and the proximal end of the second lever arm may be configured to be pinched together toward a longitudinal axis of the distal connector to remove the first protrusion and the second protrusion from the at least one catch.

In some embodiments, the at least one catch may include an annular stepped surface. In some embodiments, an entirety of an outer surface of the blunt cannula may be spaced apart from the cover, which may allow the cover to slide off the distal connector when the first lever arm and the second lever arm are released, facilitating easy removal of the cover, which may slide off the distal connector. In some embodiments, the annular wall may include a proximal end and a distal end. In some embodiments, the proximal end of the annular wall may form a proximal opening. In some embodiments, a lumen may extend from the proximal opening to and/or through the distal end of the annular wall. In some embodiments, the cover may include an inner surface forming the lumen.

In some embodiments, in response to orienting the blunt cannula to be perpendicular to the ground and pinching the proximal end of the first lever arm and the proximal end of the second lever arm together toward the longitudinal axis of the distal connector such that the first protrusion and the second protrusion are removed from the at least one catch, the cover may be configured to fall off of the distal connector under force of gravity. Thus, in some embodiments, a user may remove the cover from the distal connector with a single hand.

In some embodiments, the outer surface of the annular wall may include a ramp proximal to the at least one catch and extending outwardly in a distal to proximal direction, which may facilitate pulling the cover distally off the medical device. In some embodiments, the outer surface of the annular wall may include a ramp proximal to the at least one catch and extending outwardly in a proximal to distal direction, which may facilitate snapping of the first lever arm and the second lever arm onto the at least one catch when the cover is coupled to the distal connector.

In some embodiments, the outer surface of the annular wall may include one or more protrusions distal to the at least one catch and configured for gripping. In some embodiments, the protrusions comprises a plurality of ribs generally aligned with the longitudinal axis of the distal connector. In some embodiments, the protrusions may allow two-handed removal of the cover from the distal connector. Thus, in some embodiments, the cover may be configured for both one-handed removal and two-handed removal, depending on a preference of the user or other circumstances. In some embodiments, each of the ribs may include a proximal end that is stepped or tapered inwardly, which may create space for the first protrusion and the second protrusion and facilitate coupling of the first protrusion and the second protrusion to the at least one catch.

In some embodiments, the cover may include an annular enclosure, which may extend from the distal end of the annular wall. In some embodiments, the annular enclosure may be configured to surround the first lever arm and the second lever arm. In some embodiments, the annular enclosure may be spaced apart from the annular wall by an annular channel. In some embodiments, the first lever arm and the second lever arm may be disposed within the annular channel.

In some embodiments, the annular enclosure may include an inner surface. In some embodiments, the inner surface of the annular enclosure may include a portion that is angled outwardly in a distal to proximal direction such that in response to pinching the proximal end of the first lever arm and the proximal end of the second lever arm together toward the longitudinal axis of the distal connector, the first protrusion and the second protrusion may contact the portion and cam against the portion to facilitate removal of the cover from the distal connector. In these and other embodiments, the cover may be configured to be removed from the distal connector with a single hand and/or in any orientation. In some embodiments, the annular enclosure and/or the cover may be constructed of an elastomeric material. In some embodiments, the annular enclosure and/or the cover may be constructed of plastic or another suitable material.

In some embodiments, the annular enclosure and the annular wall may be monolithically formed as a single unit. In some embodiments, a proximal end of the cover may include a flange configured for gripping. In some embodiments, the flange may be annular. In some embodiments, the annular enclosure, the annular wall, and the flange are monolithically formed as a single unit.

In some embodiments, the medical device may include a vascular access device. In some embodiments, the vascular access device may include an instrument configured to advance distally through the distal connector when the cover is removed from the distal connector. In some embodiments, the instrument may include a tube, a guidewire, or another suitable instrument. In some embodiments, the distal connector may be configured to couple to a catheter assembly when the cover is removed from the distal connector.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is a cross-sectional view of an example distal end of a medical device, according to some embodiments;

FIG. 1B is an upper perspective view of the distal end of the medical device, according to some embodiments;

FIG. 1C is an upper perspective view of an example cover of the medical device, according to some embodiments;

FIG. 2A is an upper perspective view of the distal end of the medical device, illustrating the cover having an example annular enclosure, according to some embodiments;

FIG. 2B is a cross-sectional view of the distal end of the medical device, illustrating the annular enclosure, according to some embodiments;

FIG. 2C is a cross-sectional view of the distal end of the medical device, illustrating an example flange, according to some embodiments; and

FIG. 3 is a cross-sectional view of the distal end of the medical device, according to some embodiments.

### DETAILED DESCRIPTION

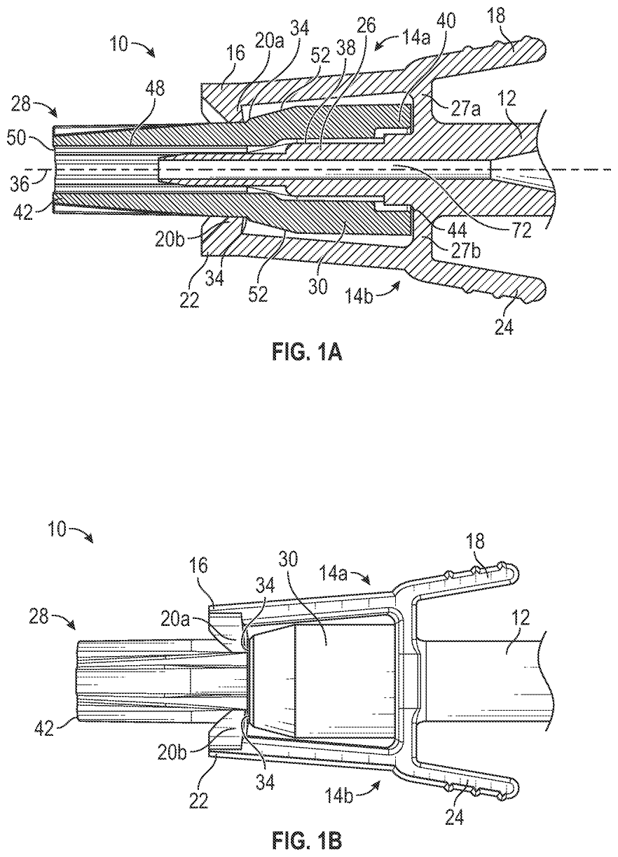

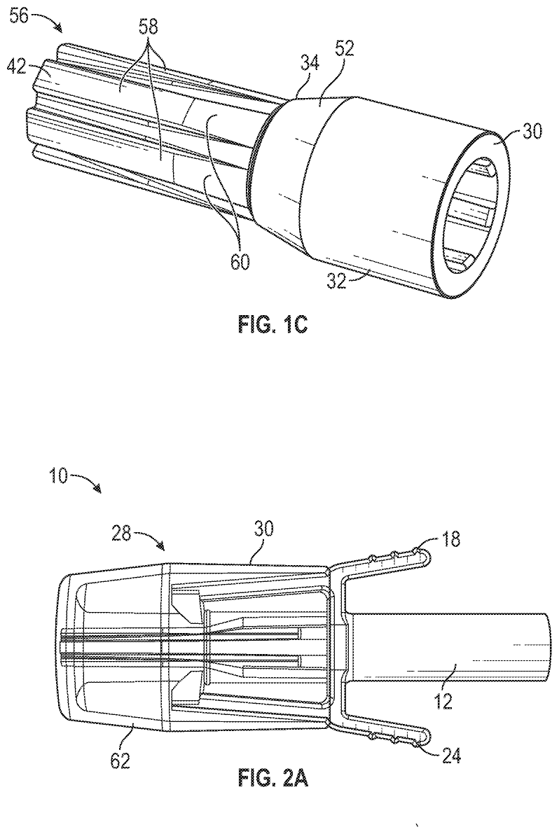

Referring now to FIGS. 1A-1C, in some embodiments, a medical device 10 may include a distal connector 12 disposed at a distal end of the medical device 10. In some embodiments, the distal connector 12 may include a first lever arm 14a, which may include a distal end 16 and a proximal end 18. In some embodiments, the distal end 16 of the first lever arm 14a may include a first protrusion 20a. In some embodiments, the distal connector 12 may include a second lever arm 14b opposing the first lever arm 14a. In some embodiments, the second lever arm 14b may include a distal end 22 and a proximal end 24. In some embodiments, the distal end 22 of the second lever arm 14b may include a second protrusion 20b. In some embodiments, the distal connector 12 may include a blunt cannula 26 disposed between the first lever arm 14a and the second lever arm 14b. In some embodiments, the first lever arm 14a and the second lever arm 14b may be flexible and configured to pivot with respect to an extension 27a and an extension 27b, respectively.

In some embodiments, the medical device 10 may include a cover 28 coupled to the distal connector 12. In some embodiments, the cover 28 may include an annular wall 30 surrounding the blunt cannula 26, which may include entirely surrounding the blunt cannula 26. In some embodiments, an outer surface 32 of the annular wall 30 may include at least one catch 34. In some embodiments, the first protrusion 20a and the second protrusion 20b may contact the at least one catch 34, which may secure the cover 28 to the distal connector 12. In some embodiments, the at least one catch 34 may be configured to prevent the distal connector 12 from moving proximally with respect to the cover 28 and the cover 28 moving distally with respect to the distal connector 12.

In some embodiments, the proximal end 18 of the first lever arm 14a and the proximal end 24 of the second lever arm 14b may be configured to be pinched together toward a longitudinal axis 36 of the distal connector 12 to remove the first protrusion 20a and the second protrusion 20b from the at least one catch 34. In further detail, in some embodiments, in response to the proximal end 18 of the first lever arm 14a and the proximal end 24 of the second lever arm 14b being pinched together towards the longitudinal axis 36, the distal end 16 of the first lever arm 14a and the distal end 22 of the second lever arm 14b may be biased outwardly such that the first protrusion 20a and the second protrusion 20b are spaced apart from the at least one catch 34, and the cover 28 is released. In some embodiments, the first lever arm 14a and the second lever arm 14b may be configured to naturally return from an outwardly biased position to an inward position in which the first protrusion 20a and the second protrusion 20b are configured to contact the at least one catch 34.

In some embodiments, the at least one catch 34 may correspond to a single catch, which may be contacted by both the first protrusion 20a and the second protrusion 20b. In these and other embodiments, the at least one catch 34 may include an annular stepped surface or an annular groove. In some embodiments, the at least one catch 34 may include a first catch and a second catch. In these embodiments, the first protrusion 20a may contact the first catch and the second protrusion 20b may contact the second catch. In some embodiments, the first catch and the second catch may each include a stepped surface or a groove.

In some embodiments, an entirety of an outer surface 38 of the blunt cannula 26 may be spaced apart from the cover 28 such that there is no interference between the outer surface 38 of the blunt cannula 26 and the cover 28. In some embodiments, this may allow the cover 28 to slide off the distal connector 12 when the first lever arm 14a and the second lever arm 14b are released, facilitating easy removal of the cover 28.

In some embodiments, the annular wall 30 may include a proximal end 40 and a distal end 42. In some embodiments, the proximal end 40 of the annular wall 30 may form a proximal opening 44 into which the blunt cannula 26 may be inserted. In some embodiments, a lumen 46 may extend from the proximal opening 44 to and/or through the distal end 42 of the annular wall 30. In some embodiments, the cover 28 may include an inner surface 48 forming the lumen 46. In some embodiments, the distal end 42 of the annular wall 30 may form a distal opening 50 or may be closed. In some embodiments, the outer surface 38 of the blunt cannula 26 may include one or more stepped surfaces, and the inner surface 48 may include one or more corresponding stepped surfaces such that a space between the blunt cannula 26 and the cover 28 is maintained.

In some embodiments, in response to orienting the blunt cannula 26 to be perpendicular to the ground and pinching the proximal end 18 of the first lever arm 14a and the proximal end 24 of the second lever arm 14b together toward the longitudinal axis 36 of the distal connector 12 such that the first protrusion 20a and the second protrusion 20b are removed from the at least one catch 34, the cover 28 may be configured to fall off of the distal connector 12 under force of gravity. Thus, in some embodiments, a user may remove the cover 28 from the distal connector 12 with a single hand. In some embodiments, the outer surface 32 of the annular wall 30 may include a ramp 52 proximal to the at least one catch 34 and extending outwardly in a distal to proximal direction, which may facilitate pulling the cover 28 distally off the medical device 10. In some embodiments, a spring force of the first lever arm 14a and the second lever arm 14b may hold the cover 28 in place, but in response to pulling the cover 28 distally with enough force, the ramp 42 may cam the first lever arm 14a and the second lever arm 14b outwardly. Thus, in some embodiments, the cover 28 may be removed without pinching the proximal end 18 of the first lever arm 14a and the proximal end 24 of the second lever arm 14b. In some embodiments, the ramp 52 may be annular.

In some embodiments, the outer surface 32 of the annular wall 30 may include one or more protrusions 56 distal to the at least one catch 34 and configured for gripping. In some embodiments, each of the protrusions 56 may include a rib 58 generally aligned with the longitudinal axis 36 of the distal connector 12. In some embodiments, the protrusions 56 may allow two-handed removal of the cover 28 from the distal connector 12. Thus, in some embodiments, the cover 28 may be configured for both one-handed removal and two-handed removal, depending on a preference of the user or other circumstances. In some embodiments, the outer surface 32 may be smooth and/or may not include the protrusions 56 such that the cover 28 is configured primarily for one-handed removal from the distal connector 12. In some embodiments, each of the ribs 58 may include a proximal end 60 that is stepped or tapered inwardly, which may create space for the first protrusion 20a and the second protrusion 20b and may facilitate coupling of the first protrusion 20a and the second protrusion 20b to the at least one catch 34.

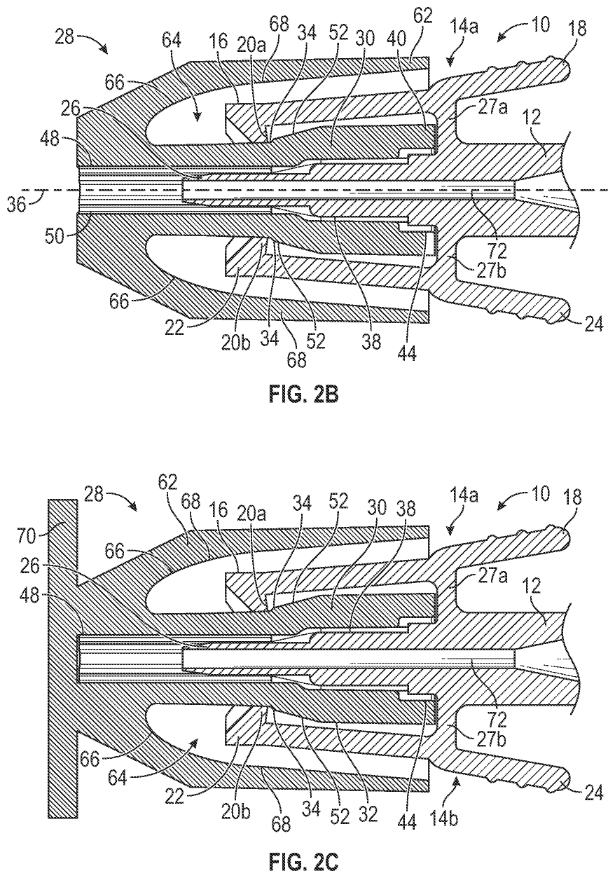

Referring now to FIGS. 2A-2C, in some embodiments, the cover 28 may include an annular enclosure 62, which may extend from the distal end 42 of the annular wall 30. In some embodiments, the annular enclosure 62 may be configured to surround the first lever arm 14a and the second lever arm 14b. In some embodiments, the annular enclosure 62 may be spaced apart from the annular wall 30 by an annular channel 64. In some embodiments, the first lever arm 14a and the second lever arm 14b may be disposed within the annular channel 64.

In some embodiments, the annular enclosure 62 may include an inner surface 66. In some embodiments, the inner surface 66 of the annular enclosure 62 may include a portion 68 that is angled outwardly in a distal to proximal direction such that in response to pinching the proximal end 18 of the first lever arm 14a and the proximal end 24 of the second lever arm 14b together toward the longitudinal axis 36 of the distal connector 12, the first protrusion 20a and the second protrusion 20b may contact the portion 68 and cam against the portion 68 to cause removal or distal movement of the cover 28 from the distal connector 12. In further detail, in some embodiments, in response to pinching the proximal end 18 of the first lever arm 14a and the proximal end 24 of the second lever arm 14b together toward the longitudinal axis 36 of the distal connector 12, the first protrusion 20a and the second protrusion 20b may contact the portion 68 and slide along the portion 68, which may force the cover 28 distally and may result in removal of the cover 28 from the distal connector 12. In these and other embodiments, the cover 28 may be configured to be removed from the distal connector 12 with a single hand and/or in any orientation. In some embodiments, the portion 68 may extend to a proximal end of the annular enclosure 62.

In some embodiments, the annular enclosure 62 and/or the cover 28 may be constructed of an elastomeric material. In some embodiments, the annular enclosure 62 and/or the cover 28 may be constructed of plastic or another suitable material. In some embodiments, the annular enclosure 62 and the annular wall 30 may be monolithically formed as a single unit.

As illustrated in FIG. 2C, in some embodiments, a proximal end of the cover 28 may include a flange 70 configured for gripping. In some embodiments, the flange 70 may be annular. In some embodiments, one or more of the following may be monolithically formed as a single unit: the annular enclosure 62, the annular wall 30, and the flange 70.

In some embodiments, the medical device 10 may include a vascular access device. In some embodiments, the vascular access device may include an instrument, which may be configured to extend into a catheter assembly and/or through the catheter assembly into vasculature of a patient. In some embodiments, when the cover 28 is removed from the distal connector 12, the instrument may be configured to advance distally through a lumen 72 extending through the distal connector 12. In some embodiments, the instrument may include a blood collection tube, a guidewire, or another suitable instrument. In some embodiments, the distal connector 12 may be configured to couple to the catheter assembly when the cover 28 is removed from the distal connector 12. Some examples of the medical device 10 may be further described in U.S. patent application Ser. No. 17/574,127, filed Jan. 12, 2022, and U.S. patent application Ser. No. 17/701,124, filed Mar. 22, 2022, which are both hereby incorporated by reference in their entirety. As mentioned, a type of the medical device 10 may vary, according to some embodiments.

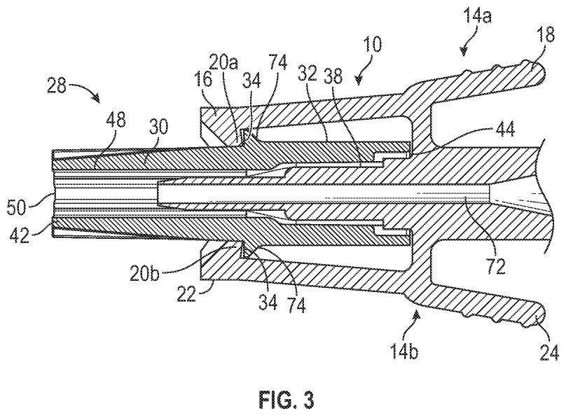

Referring now to FIG. 3, in some embodiments, the outer surface 32 of the annular wall 30 may include a ramp 74 proximal to the at least one catch 34 and extending outwardly in a proximal to distal direction, which may facilitate pushing the cover 28 proximally onto the medical device 10 during assembly and/or after use of the medical device and/or snapping of the first lever arm 14a and the second lever arm 14b onto the at least one catch 344 when the cover 28 is being coupled to the distal connector 12. In some embodiments, the ramp 74 may be annular.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A medical device, comprising:
a distal connector, comprising:
a first lever arm, comprising a distal end and a proximal end, wherein the distal end of the first lever arm comprises a first protrusion;
a second lever arm opposing the first lever arm, the second lever arm comprising a distal end and a proximal end, wherein the distal end of the second lever arm comprises a second protrusion; and
a blunt cannula disposed between the first lever arm and the second lever arm; and
a cover coupled to the distal connector and comprising an annular wall surrounding the blunt cannula, wherein an outer surface of the annular wall comprises at least one catch, wherein the first protrusion and the second protrusion contact the at least one catch, wherein the proximal end of the first lever arm and the proximal end of the second lever arm are configured to be pinched together toward a longitudinal axis of the distal connector to remove the first protrusion and the second protrusion from the at least one catch.

2. The medical device of claim 1, wherein the at least one catch comprises an annular stepped surface.

3. The medical device of claim 1, wherein an entirety of an outer surface of the blunt cannula is spaced apart from the cover.

4. The medical device of claim 1, wherein the annular wall comprises a proximal end and a distal end, wherein the proximal end of the annular wall forms a proximal opening, wherein a lumen extends from the proximal opening to the distal end of the annular wall, wherein the cover comprises an inner surface forming the lumen.

5. The medical device of claim 1, wherein in response to orienting the blunt cannula to be perpendicular to the ground and pinching the proximal end of the first lever arm and the proximal end of the second lever arm together toward the longitudinal axis of the distal connector such that the first protrusion and the second protrusion are removed from the at least one catch, the cover is configured to fall off of the distal connector under force of gravity.

6. The medical device of claim 1, wherein the outer surface of the annular wall comprises a ramp proximal to the at least one catch and extending outwardly in a distal to proximal direction.

7. The medical device of claim 1, wherein the outer surface of the annular wall comprises a ramp proximal to the at least one catch and extending outwardly in a proximal to distal direction.

8. The medical device of claim 1, wherein the outer surface of the annular wall comprises a plurality of protrusions distal to the at least one catch and configured for gripping.

9. The medical device of claim 8, wherein the protrusions comprises a plurality of ribs generally aligned with the longitudinal axis of the distal connector.

10. The medical device of claim 9, wherein each of the plurality of ribs comprises a proximal end that is stepped or tapered inwardly.

11. The medical device of claim 1, wherein the cover further comprises an annular enclosure extending from the distal end of the annular wall and configured to surround the first lever arm and the second lever arm, wherein the annular enclosure is spaced apart from the annular wall by an annular channel, wherein the first lever arm and the second lever arm are disposed within the annular channel.

12. The medical device of claim 11, wherein the annular enclosure comprises an inner surface, wherein the inner surface of the annular enclosure comprises a portion that is angled outwardly in a distal to proximal direction such that in response to pinching the proximal end of the first lever arm and the proximal end of the second lever arm together toward the longitudinal axis of the distal connector, the first protrusion and the second protrusion contact the portion and cam against the portion to facilitate removal of the cover from the distal connector.

13. The medical device of claim 12, wherein the enclosure is constructed of an elastomeric material.

14. The medical device of claim 11, wherein the annular enclosure and the annular wall are monolithically formed as a single unit.

15. The medical device of claim 11, wherein a proximal end of the cover comprises a flange configured for gripping.

16. The medical device of claim 15, wherein the flange is annular.

17. The medical device of claim 15, wherein the annular enclosure, the annular wall, and the flange are monolithically formed as a single unit.

18. The medical device of claim 1, wherein the medical device comprises a vascular access device, wherein the vascular access device comprises an instrument configured to advance distally through the distal connector when the cover is removed from the distal connector.

19. The medical device of claim 18, wherein the instrument comprises a tube or a guidewire.

20. The medical device of claim 1, wherein the distal connector is configured to couple to a catheter assembly when the cover is removed from the distal connector.
